(19) United States
(12) Patent Application Publication
(10) Pub. No.: US 2014/0302099 A1
(43) Pub. Date: Oct. 9, 2014

(54) Ophthalmic Composition Containing Cyclosporine And method For Preparing Same

(71) Applicant: Yong Nam KIM, (US)

(72) Inventor: Yong Nam Kim, Daejeon (KR)

(21) Appl. No.: 14/351,099

(22) PCT Filed: Oct. 9, 2012
(86) PCT No.: PCT/KR2012/008160

§ 371 Date: Apr. 10, 2014

(30) Foreign Application Priority Data
Oct. 10, 2011 (KR) 10-2011-0102909

(51) Int. Cl. A61K 47/10; A61K 47/02; B01J 19/08; A61K 38/13

(52) U.S. Cl. CPC A61K 47/10; A61K 38/13; A61K 47/02; B01J 19/08
USPC 424/278.1; 204/157.71

(57) ABSTRACT

The present invention relates to an ophthalmic composition containing cyclosporine as an active ingredient and comprising propylene glycol, a surfactant, and purified water, and to a method for preparing same. The ophthalmic composition according to the present invention is in the form of a clear and transparent solution, and increases patient compliance due to the absence of side effects, such as cloudy vision or a scorching sensation, and has a high bioavailability due to improved cyclosporine absorbance in the ocular tissue, which is the target organ.

### TECHNICAL FIELD

The present invention relates to an ophthalmic composition containing cyclosporin, particularly to the ophthalmic composition containing cyclosporin which is in the form of a clear and transparent solution without precipitation of cyclosporin and which causes no irritation to the eye when applied, and the method for preparing the same.

### BACKGROUND ART

Cyclosporin is a material known to be an immunosuppressive agent. It stimulates the lacrimal gland to promote tear secretion and thus is very effective in the treatment of dry eye (keratoconjunctivitis sicca) (see U.S. Pat. No. 4,839,342).

Cyclosporin is a representative insoluble drug, and it is known that an aqueous preparation containing cyclosporin as an active ingredient is generally difficult to manufacture (see U.S. Pat. No. 5,051,402). Accordingly, formulations in which cyclosporin is dissolved in oily components such as olive oil, corn oil, etc. were developed (see U.S. Pat. No. 4,839,342). However, oil-containing formulations cause severe irritation or blurring vision when applied to the eye, and thus their effectiveness as eye drops is limited. A preparation in which cyclosporin was dissolved in an ethanol-containing oily solution was proposed, but the preparation had a problem in that when it was contacted with water, cyclosporin was precipitated and thus its bioavailability rapidly decreased. To prevent the precipitation of cyclosporin, polyoxyethylated caster oil was used as a solubilizing agent. However, its problem regarding safety was pointed out as described in U.S. Pat. No. 5,051,402.

Now, Restasis™ (Allergan) is commercially available worldwide as a cyclosporin-containing ophthalmic composition for the treatment of dry eye. The formulation uses castor oil as a solubilizing agent for cyclosporin and is an emulsion formulation formed by use of crosslinked polyacrylate as a dispersing agent and an emulsifier such as polysorbate 80 (see KR Patent No. 368,181). As is disclosed in this literature, the simultaneous use of an emulsifier and a dispersing agent may alleviate eye irritation. However, the formulation may have side effects such as a burning sensation due to the emulsion particles and castor oil used as a solubilizing agent, and its patient compliance is low. Moreover, safety issues are noted because castor oil may include ricin, which is listed among Class B toxins by the United States Center for Disease Control and Prevention.

Accordingly, there is a need for a new ophthalmic composition containing cyclosporin as an active ingredient in which cyclosporin does not precipitate, alleviates side. effects such as a burning sensation and eliminates the problem of toxicity, and thus which can be safely applied.

### LIST OF PRIOR ART DOCUMENTS

1. U.S. Pat. No. 4,839,342 (published on Jun. 13, 1989)
2. U.S. Pat. No. 5,051,402 (published on Sep. 24, 1991)
3. KR Patent No. 368,181 (published on Jul. 3, 1997)

### CONTENTS OF THE INVENTION

### Problems to be Solved

The object of the present invention is the provision of a clear and transparent ophthalmic composition which has improved absorption of cyclosporin into the ocular tissue without causing irritation to the eyes and produces no precipitate even when contacted with water or over a long period of storage, and a manufacturing method thereof.

### Technical Means

To achieve the above object, the present invention provides an ophthalmic composition comprising cyclosporin as an active ingredient, propylene glycol, emulsifier and purified water.

In the present invention, cyclosporin is a cyclic peptide consisting of 11 amino acids and having a phramacologically useful immunosuppressive action. Cyclosporins used in the present invention are cyclosporin A, B, C, D or G which are classified according to the structures of the constituting amino acid residues, or analogues thereof. Preferably used is cyclosporin A whose clinical examples and pharmacological activity are most elucidated.

In the present ophthalmic composition, propylene glycol (PG) is used as a solubilizing agent for cyclosporin. In the present ophthalmic composition, instead of higher fatty acid glycerides such as castor oil or corn oil which are conventionally used as solubilizing agents for cyclosporin but have a high risk of irritating sensitive organs such as the eye, propylene glycol is the first choice as a solvent to dissolve cyclosporin rapidly and completely, and form a clear and transparent cyclosporin-containing solution. In the present ophthalmic composition, the weight ratio of cyclosporin to propylene glycol is preferably, but not limited to, 1:35 to 45. Even outside the above range the desired effect can be obtained, but if the ratio is less than 1:35, cyclosporin may not be completely dissolved. In case the ratio is more than 1:45, stability of the composition can be reduced, and therefore the above. range is most preferable.

In the present ophthalmic composition, surfactants are used to enhance the stability of the composition. Preferably polysorbate is used, and most preferably polysorbate 80 is used. In the present ophthalmic composition, the weight ratio of cyclosporin to surfactant is preferably, but not limited to, 1:15 to 25. Even outside the above range, the desired effect can be obtained, but the composition is most stable within the range.

The present ophthalmic composition may further include pH-adjusting agents which are conventionally used in ophthalmic preparations. As such pH-adjusting agents, sodium hydroxide, sodium phosphate and the like can be used; in particular the mixture of Na₂PO₄ and NaH₂PO₄ is preferred. If required, buffering agents such as phosphate, citrate, acetate or borate may he included, When needed, an isotonic agent conventionally used in ophthalmic preparations such as NaCl may be further included.

The rest of the present ophthalmic composition comprises purified water. The purified water used in the present invention is preferably sterile purified water or injectable distilled water to which a high-frequency wave is applied. Most preferably, the purified water is cooled to 2-10° C., applied with a high-frequency wave and then used. When the cyclosporin in propylene glycol is diluted with the purified water applied with a high-frequency wave, the stability of the solution is dramatically increased, and this is one of the key technical features of the present invention.

A high-frequency wave at 900 to 3000 mhz is applied to the water for about 5 to 30 minutes. The present inventor found that when the cyclosporin solution in the propylene glycol is mixed with purified water applied with a high-frequency wave, the physical stability of the resulting solution was dramatically increased compared to a resulting solution formed by mixing with ordinary purified water. For example, when the solution was mixed with ordinary purified water, some precipitates were produced in the resulting solution in about three days, and the physical stability of the resulting solution was very low. However, when mixed with purified water treated with a high-frequency wave the resulting solution maintained its physical stability after a long-term storage of more than nine months.

In another aspect, a method of preparing the present ophthalmic composition is provided.

The method comprises the following steps:
(a) applying a high-frequency wave to purified water to obtain purified water applied with a high-frequency wave;
(b) dissolving cyclosporin in propylene glycol and adding a surfactant to obtain a first solution;
(c) diluting the first solution obtained in step (b) with the purified water applied with a high-frequency wave with adding shearing force,

In step (a), a 900˜3000 MHz high-frequency wave is applied to the purified water for about 5 to 30 minutes. At that time, it is more preferable to cool the purified water to 2-10° C. and then apply the high-frequency wave to the cooled water to enhance stability.

In step (b), cyclosporin is dissolved in propylene glycol, and then a surfactant is further added to promote the stability of the prepared solution.

In step (c), the cyclosporin-containing propylene glycol solution (the first solution) prepared in step (b) is diluted with the purified water treated with a high-frequency wave applying shearing force. At this time, the shearing force is preferably applied by stirring the solution at about 3000˜5000 rpm. Specifically, the first solution and the purified water are mixed for about 1 to 2 minutes while stirring at about 1000˜5000 rpm. And then further shearing force is applied by continuing strong stirring at about 3000˜5000 rpm for about 5 to 10 minutes, and then the solution is allowed to stand for about 4 to 6 minutes. Such stirring and standing processes can be repeated, preferably three times. So far, in the conventional solubilizing process for an insoluble drug, stirring is usually done at less than 500˜700 rpm. However, in the present invention vigorous stirring at about 3000˜5000 rpm is applied. It is believed that vigorous stirring allows the cyclosporin-containing propylene glycol solution to be evenly dispersed in water and the resulting solution becomes a colloidal state (water-to-water state), and thus its stability is dramatically improved. Applying a high-frequency wave to purified water in advance is a process that activates the water in order to maintain such a colloidal state.

The present inventor found that the physical and storage stabilities are dramatically increased by using purified water treated with a high-frequency wave as water to be mixed with cyclosporin-propylene glycol solution and by adding further shearing force to the obtained mixed solution of cyclosporin-propylene glycol-purified water.

The present ophthalmic composition comprising the solution of cyclosporin-propylene glycol-purified water prepared by the present method maintains a form of clear and transparent solution without precipitation when it contacts water or bodily fluids, or even during long-term storage. Its physical and storage stabilities are dramatically increased. In addition, the present composition includes no oil components such as castor oil which may irritate the eye, and thus it can provide cyclosporin-containing ophthalmic drugs without side effects such as a stinging or burning sensation. Moreover, the present ophthalmic composition shows excellent transport of cyclosporin to eye tissues such as the conjunctiva and improves the absorption of cyclosporin to the eye tissues.

Hereinafter, the present invention is explained in more detail by the following Examples. However, the scope of the present invention is not limited thereby.

### Effect of the Invention

The present cyclosporin-containing ophthalmic composition is in the form of a clear and transparent solution, which generates no precipitate of cyclosporin after contacting with water or bodily fluids, or a long storage period. It has no side effects such as blurred vision or a burning sensation, and thus patient compliance is improved. The absorption of cyclosporin into the target eye tissue and thereby bioavailability are improved.

### BRIEF DESCRIPTION OF THE DRAWINGS

FIG. 1 is a photograph showing the physical property of the present composition compared to that of the commercially available eye drop formulation of Restasis™ as a reference preparation.

FIG. 2 shows a residual amount of cyclosporin A in ocular tissues of the present composition compared to that of the commercially available eye drop formulation of Restasis™ as a reference preparation.

### CONCRETE EXPLANATION TO CARRY OUT THE INVENTION

### EXAMPLE 1

### The Manufacture of the Present Ophthalmic Composition

100 L of distilled water for injection was placed in a preparation tank (capacity: 100-150 L), applied with 2400 mhz of a high-frequency wave for 15 minutes at 4° C. and then allowed to stand to obtain the purified water applied with a high-frequency wave. 2000 g of propylene glycol was added to a stirring vessel (capacity: 5 L), and 50 g of cyclosporin A was added thereto. The solution was stirred for 15 minutes at 400˜500 rpm until the cyclosporin A was completely dissolved. 1000 g of polysorbate 80 was added to the solution under continuous stirring to obtain a stock solution. To 48.5 L of the purified water applied with a high-frequency wave in the tank, the stock solution was added while stirring at 4000 rpm. The mixture was stirred for 10 minutes at 4000 rpm and then allowed to stand for 5 minutes. This stirring-and-standing process was repeated three times, The pH of the resulting solution was adjusted to 7-8 by adding 14 g of NaCl, 42 g of Na₂HPO4 and 14 g of NaH₂PO₄, and the resulting solution was filtered via 0.2 μm of filter paper to prepare the present ophthalmic composition.

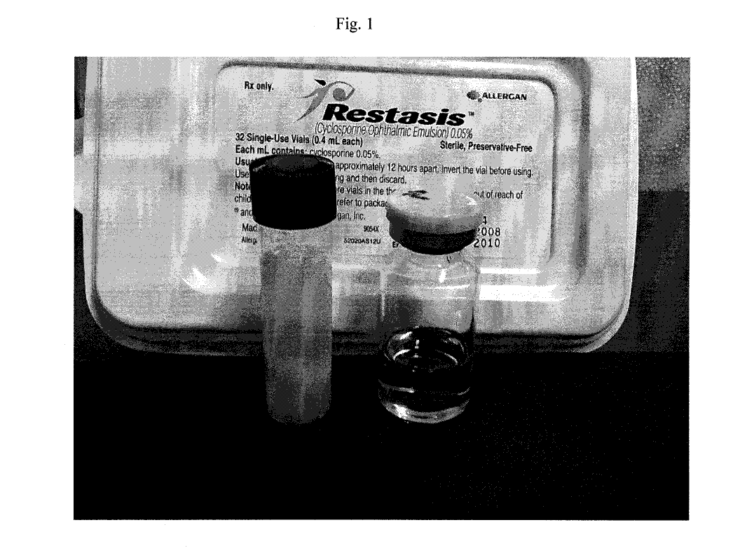

The prepared present ophthalmic composition is shown in FIG. 1 with commercially available Restasis™ eye drops 0.05% purchased from Samil Allergan, Korea. The product in the left test tube is the commercially available Restasis™ eye drops 0.05%, and the product in the right test tube is the present ophthalmic composition. As may be clearly seen in the photo of FIG. 1, the present ophthalmic composition is a clear and transparent solution whose visible light transmittance is very high and thus addresses the shortcomings of the reference preparation, such as blurred vision, discomfort and the like due to the low transmittance of visible light. The present ophthalmic composition has no particles of a size that could irritate the eye.

### COMPARATIVE EXAMPLE 1

The Comparative Example 1 preparation was prepared by the method described in Example 1 except that ordinary purified water was used rather than the purified water treated with the high-frequency wave.

### EXPERIMENTAL EXAMPLE 1

To investigate storage stability of the present composition, the compositions of Example 1 and Comparative Example 1 were stored for 70 days or 270 days at room temperature or under refrigeration, and then the state of the compositions was observed and the titre of cyclosporin A was measured by HPLC. HPLC operating conditions are as follows, and the results are shown in Table 1.

HPLC operating conditions:

Column: Nova-Pak phenyl 4 μm (3.9×150 mm),

flow rate: 1 μl/min,

pump pressure: 700-800 psi,

detection wavelength: 205 nm,

mobile phase: acetonitrile (65%) +0.04 M mono basic phosphate buffer (35%),

injection volume: 30 μl

TABLE 1

| | Example 1 composition | | Comparative Example 1 composition | |
|---|---|---|---|---|
| | State | titre of cyclosporin A | State | titre of cyclosporin A |
| Immediately after | Clear and | 0.5 mg/ml | Clear and | 0.5 mg/ml |
| preparation | transparent | | transparent | |
| | solution | | solution | |
| After 70 days | Clear and | 0.493 mg/ml | Precipitation | Impossible to measure |
| storage at room | transparent | | | the titre |
| temperature | solution | | | |
| After 270 days | Clear and | 0.492 mg/ml | Precipitation | Impossible to measure |
| storage at room | transparent | | | the titre |
| temperature | solution | | | |
| After 70 days | Clear and | 0.498 mg/ml | Precipitation | Impossible to measure |
| storage under | transparent | | | the titre |
| refrigeration | solution | | | |
| After 270 days | Clear and | 0.493 mg/ml | Precipitation | Impossible to measure |
| storage under | transparent | | | the titre |
| refrigeration | solution | | | |

As can be seen from the results in the above Table 1, the present composition maintained its physical property of a clear and transparent solution for a long storage period of at least nine months at room temperature and under refrigeration. The titre of cyclosporin A remained approximately the same. The storage stability of the present composition was confirmed. On the other hand, precipitation occurred in three days at room temperature in the Comparative Example 1 preparation. From these results, it is confirmed that the storage stability of the present composition was dramatically increased by using purified water applied with a high-frequency wave.

### EXPERIMENTAL EXAMPLE 2

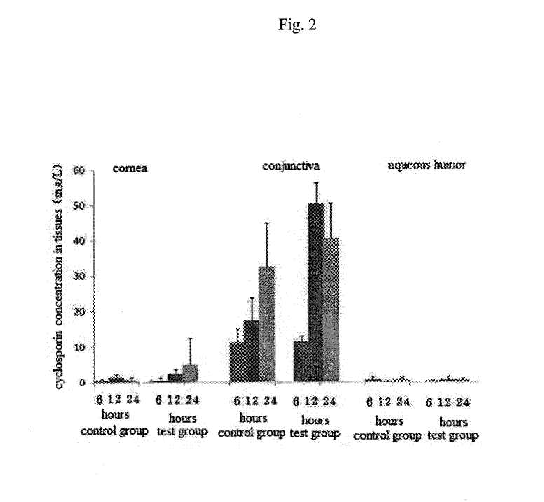

2˜3 kg of New Zealand albino rabbits (n=54) were purchased, kept in quarantine and acclimated in the breeding room amid constant temperature and humidity (23±3, 50±10%) for 5 days, and then used for a test. The composition obtained in Example 1 was applied inside the lower eyelid of each rabbit of a test group at a dose of 2 μg of cyclosporin A per 1 kg of body weight. The eyes of the rabbits were manually closed for 5 seconds. After 6, 12 and 24 hours, the rabbits were euthanized and their ocular tissues were extracted. For measurement of the residual cyclosporin A in the eye tissues, a radioisotope-labeled cyclosporin A was measured using liquid scintillation. To rabbits of the control group, commercially available Restasis™ eye drops 0.05% was applied in the same way as described above. The residual amounts of cyclosporin A in the eye tissues of the rabbits of the control group and the test group are shown in FIG. 2. As shown in FIG. 2, it is confirmed that the present ophthalmic composition has equal or superior effect compared to the reference preparation in respect to the residual amounts of cyclosporin A in all ocular tissues.

1. A non-irritating ophthalmic composition comprising cyclosporin as an active ingredient, propylene glycol, surfactant and purified water.

2. The non-irritating ophthalmic composition according to claim 1, wherein the surfactant is polysorbate 80.

3. The non-irritating ophthalmic composition according to claim 1 wherein the purified water is the purified water applied with a high-frequency wave.

4. The non-irritating ophthalmic composition according to claim 1, wherein the cyclosporin is cyclosporin A.

5. The non-irritating ophthalmic composition according to claim 1, wherein the weight ratio of cyclosporin to propylene glycol is 1:35 to 45.

6. The non-irritating ophthalmic composition according to claim 1, wherein the weight ratio of cyclosporin to surfactant is 1:15 to 25.

7. The non-irritating ophthalmic composition according to claim 1, wherein a pH-adjusting agent is further comprised.

8. The method of preparing a cyclosporin-containing ophthalmic preparation comprising the following:
(a) applying a high-frequency wave to purified water to obtain purified water applied with a high-frequency wave;
(b) dissolving cyclosporin in propylene glycol and adding a surfactant to obtain a first solution;
(c) diluting the first solution obtained in step (b) with the purified water applied with a high-frequency wave with adding shearing force.

9. The method according to claim 9, wherein 900˜3000 mhz of a high-frequency wave is applied in step (a).

10. The method according to claim 9, wherein shearing force is added by stirring at 3000˜5000 rpm in step (c).
